(19) United States
(12) Patent Application Publication
(10) Pub. No.: US 2020/0022480 A1
(43) Pub. Date: Jan. 23, 2020

(54) DEVICE FOR PACKAGING AND DISPENSING A COSMETIC COMPOSITION

(71) Applicant: CHANEL PARFUMS BEAUTE, NEUILLY SUR SEINE (FR)

(72) Inventors: Alexandre LAUER, PANTIN CEDEX (FR); Sylvie PIRES LEITAO, PANTIN CEDEX (FR); Nicolas CASTEX, NEUILLY (FR)

(73) Assignee: CHANEL PARFUMS BEAUTE, NEUILLY SUR SEINE (FR)

(21) Appl. No.: 16/481,604

(22) PCT Filed: Jan. 24, 2018
(86) PCT No.: PCT/FR2018/050165

§ 371 Date: Jul. 29, 2019

(30) Foreign Application Priority Data
Jan. 30, 2017 (FR) 17 50749

(51) Int. Cl. A45D 40/08; A61K 8/02; A61K 8/06; A61K 8/04; A61K 8/31; A61K 8/58; A61Q 1/02

(52) U.S. Cl. CPC A45D 40/08; A61K 8/0216; A61K 8/062; A61K 8/042; A61K 2800/522; A61K 8/585; A61Q 1/02; A61K 2800/436; A61K 8/31

(57) ABSTRACT

A device for packaging and dispensing a cosmetic composition, including a housing receiving a cosmetic composition having a hardness of greater than or equal to 20 g, and at least one grid arranged for the passage of the cosmetic composition through the grid and having through-orifices. The device is configured such that the cosmetic composition undergoes a reduction in its hardness of greater than 50%, preferably greater than 70%, during the passage of the cosmetic composition through the at least one grid.

The present invention relates to the cosmetics field and more particularly to a device for packaging and dispensing a cosmetic product.

The cosmetic compositions according to the invention can in particular pertain to the makeup, care or fragrancing compositions field. Among other makeup products, mention may be made of lipsticks, foundations, complexion correctors, complexion bases, concealers, blushers, highlighters, eye shadows, and more generally any body makeup product. The care products may be lip balms, moisturizing care products, antiwrinkle care products, antisun care products.

Products with a pasty to solid consistency at ambient temperature, intended in particular for application to the skin, are generally packaged either in the form of a stick—or baton, or in containers such as jars or pots. The latter then require the use of an applicator, such as a sponge or a brush, which does not allow the consumer to carry their product and to use it in all circumstances.

Cosmetic compositions in stick form, such as lipsticks, are widely known and are regularly used by numerous consumers in the largest fields of bodycare. Sticks are desirable for their ease of application and their portability.

However, they can have the drawback of being perceived as too hard when they are applied to the skin. Conversely, they can lose their mechanical and esthetic properties under the effect of temperature variations for example. This has the effect of limiting their attraction and their strength during application, which can result in dislodging or breaking of the stick under the effect of the pressure exerted by the consumer when spreading the product.

In order to use such cosmetic compositions, and in particular when it is a question of applying them for example to the skin or the lips, it is important for these compositions to have good rheological properties. In particular, they must be sufficiently melting to be easily spread on the skin without compromising the sensory properties or the comfort of the consumer, but they must also have good mechanical strength properties in order to avoid risks of the stick breaking. These constraints can=be limiting from the formulation point of view. In particular, cosmetic compositions may be loaded with pigments in order to obtain an intense color in the makeup field. Likewise, in the care field, it may be desirable to use solid or pasty fatty substances.

Other problematic aspects are sometimes the metering of the product and the unwanted tendency of certain preparations to form residues on the application surface.

One objective of the invention is thus to overcome the deficiencies of the existing means for packaging and applying cosmetic compositions and to propose a device for packaging and dispensing a cosmetic composition suitable for the application of a cosmetic composition having a certain hardness, this being without the intervention of an applicator.

To this effect, according to the invention, a device for packaging and dispensing a cosmetic product is provided for, comprising:
a housing receiving a cosmetic composition having a hardness (d) of greater than or equal to 20 g,
at least one grid arranged for the passage of the cosmetic composition through the grid and having through-orifices,

the device being configured such that the cosmetic composition undergoes a reduction of its hardness (Δd) of greater than 50%, preferably greater than 70%, during the passage of the cosmetic composition through said at least one grid.

The hardness of the cosmetic composition is evaluated at 20° C. using a TA-XT Plus Microstable System texturometer from the company Swantech.

The device according to the invention makes it possible to package and store the cosmetic composition in its initial state which can be a pasty, solid, structured and/or brittle state, while at the same time applying it in a destructured and ductile form. Indeed, when the cosmetic composition passes through the grid, its rheological properties are modified and the composition passes from a structured and sometimes brittle state to a destructured and ductile, more fluid state, resulting at the physical level in the reduction of its hardness.

Thus, the invention makes it possible to provide a means for packaging and dispensing compositions having a high hardness, which are difficult to spread directly on the skin in the native state.

The invention also makes it possible to provide a means for packaging and dispensing cosmetic compositions having too high a consistency to be delivered by means of a pump-dispenser bottle, but not having enough mechanical strength to be able to be packaged in stick form.

The invention thus makes it possible to satisfactorily dispense such compositions without particular constraints in terms of their formulation. The device according to the invention also improves the spreading, while at the same time preserving the gesture of a conventional lipstick in the case of a labial application, and providing in all cases an extremely easy application owing to the transportable nature of the device according to the invention.

Furthermore, the cosmetic composition can be masked in the device if the said device has for example opaque or not completely transparent walls, which makes it possible to dispense with the esthetic appearance of the cosmetic composition.

The device according to the invention is advantageously pleasant to use and generates a pleasant sensation to the touch, such as a soft sensation. This sensation can for example be due to the softened texture obtained after the passage of the cosmetic composition through the grid.

Advantageously, the device according to the invention makes it possible to control and modulate the amount of cosmetic composition to be applied. Indeed, only the composition having been destructured by its passage through the grid is capable of being applied. By adjusting the thickness of the film applied, it is thus possible to vary the coverage, in the case of a makeup product, or, more generally, to control the amount applied.

In the case of a cosmetic makeup composition, the shininess of the film applied to the skin can be increased by the passage through the grid(s) of the device according to the invention, which may be very advantageous for certain applications.

Grids

The device according to the invention can have a single grid or, advantageously, at least two grids one after the other in a direction of passage of the cosmetic composition through the grids. This makes it possible in particular to obtain a greater effect of guiding of the cosmetic composition, each grid directing the cosmetic composition slightly more in the direction of application. The use of several grids allows a greater and/or facilitated destructuring of the cosmetic composition. Indeed, the destructuring effects of each grid accumulate and the cosmetic composition undergoes a first destructuring during its passage—or its extrusion—through the first grid, and an additional destructuring during each subsequent passage through each additional grid.

The orifices of each grid can be superimposed facing one another, or, on the contrary, the orifices may be offset. For example, two grids can be arranged such that a corner of a through-orifice of the first grid is located opposite the center of a through-orifice of the second grid.

The through-orifices have, for example, a larger dimension of between 40 and 475 μm. Such dimensions advantageously allow the consumer not make too much effort in causing the cosmetic composition to pass through the grid(s), while at the same time allowing satisfactory destructuring.

When there are several grids, the orifices are advantageously of larger dimension decreasing in the direction of the path of the composition.

The orifices may be of any shape, and in particular of circular, oval, hexagonal, quadrilateral—in particular rectangular—or pentagonal cross section.

Advantageously, the single grid or the outermost grid of the device itself can consist of a material that is pleasant to the touch, for example a textile material, or a material that generates a sensation of freshness, for example a metallic material. Thus, the grid or at least one of the grids can advantageously be woven, for example by means of a woven formed by warp threads and weft threads, the warp threads conventionally having a diameter different than that of the weft threads.

The term “first grid” is intended to mean the first grid through which the cosmetic composition is extruded. The term “outermost grid” is intended to mean the last grid through which the cosmetic composition passes before being applied.

According to one particular embodiment, the device comprises two grids in which the first grid has orifices of larger dimension of between 160 and 475 μm, and the external grid has orifices of larger dimension of between 42 and 125 μm.

Composition

The cosmetic composition packaged and dispensed by the device according to the invention can be in a large variety of forms. It can in particular be in anhydrous form, or in the form of a simple emulsion or a multiple emulsion.

In the case of a simple emulsion, it may be an oil-in-water or water-in-oil emulsion. This means that it is a colloidal suspension of droplets of a hydrophilic solution in a hydrophobic solution, or conversely, a colloidal suspension of droplets of a hydrophobic solution in a hydrophilic solution. Such emulsions are not therefore limited strictly speaking to droplets of water in oil or of oil in water.

In the case of a multiple emulsion, there is a colloidal suspension of an emulsion in another solution that can be described as a matrix solution. If the emulsion is an oil-in-water emulsion, the matrix solution will be a hydrophobic solution and, conversely, if the emulsion is a water-in-oil emulsion, the matrix solution will be an aqueous solution.

It is possible to use emulsions of greater order, such as water-in-oil-in-water-in-oil or oil-in-water-in-oil-in-water emulsions, etc.

Such emulsions thus comprise one or more aqueous phases and/or one or more fatty phases.

The size of the droplets is conditioned by the properties of the solutions used and is sufficiently small to prevent the droplets from aggregating together. Those skilled in the art, with knowledge of colloid sciences, will know how to adjust the properties of the emulsion to the formulation of the various phases used, and reciprocally will know how to adjust the formulation of the various phases if they wish to obtain a particularly advantageous droplet size.

The composition is preferably packaged in a form chosen from a cast anhydrous composition, an aqueous gel and an oily gel.

Preferably, the cosmetic composition has at least one fatty phase structured with a fatty-phase structuring agent. The fatty phase(s) of the cosmetic composition preferably comprise 0.5% to 90% by weight of at least one fatty-phase structuring agent. According to one advantageous embodiment, the fatty-phase structuring agent can be chosen from waxes or fatty-phase gelling agents such as clays made hydrophobic or silicone elastomers, pasty fatty substances, and mixtures thereof.

Composition—Waxes

The term “wax” is intended to mean a fatty substance having a melting point of greater than 30° C. and generally less than 110° C., which is liquid under the conditions for preparing the composition and has, in the solid state, an anisotropic crystalline organization. The waxes according to the invention can be chosen from polar waxes, apolar waxes, or mixtures thereof.

The term “apolar wax” is intended to mean a wax comprising only hydrogen and carbon atoms.

The term “polar wax” is intended to mean a wax comprising at least one heteroatom such as oxygen, nitrogen, silicon or phosphorus.

The waxes that can be used may be waxes of animal, plant, mineral or synthetic origin.

Among the waxes that can be used in the present invention, mention may for example be made of hydrocarbon-based waxes comprising an alkyl chain of 10 to 60 carbon atoms. Said chain may be saturated or unsaturated, and linear or branched. The term “hydrocarbon-based wax” is intended to mean a wax essentially formed, or even consisting, of carbon and hydrogen atoms, and optionally oxygen and/or nitrogen atoms, and containing no silicon or fluorine atom. It may contain alcohol, ester, ether, carboxylic acid, amine and/or amide groups. Among the hydrocarbon-based waxes that can be used in the context of the invention, mention may in particular be made of:
fatty alcohols such as, for example, stearyl alcohol, cetearyl alcohol or mixtures thereof,
fatty acids,
fatty acid esters including triglycerides, diglycerides and monoglycerides,
ethoxylated fatty alcohols,
ethoxylated fatty acids and salts thereof,
fatty acid ethers.

Among the fatty acid esters that can be used in the context of the invention, mention may for example be made of stearyl stearate, stearyl heptanoate, stearyl caprylate, stearyl behenate, stearyl octyldodecanol, cetearyl behenate, behenyl behenate, ethylene glycol distearate, jojoba esters, and mixtures thereof.

By way of illustration of the waxes that are suitable for the invention, mention may also be made of beeswax, lanolin wax, and Chinese insect waxes, rice bran wax, carnauba wax, candelilla wax, Ouricury wax, esparto wax, berry wax, shellac wax, Japan wax and sumac wax; montan wax, orange and lemon waxes, microcrystalline waxes, paraffins and ozocerite; polyethylene waxes, and mixtures thereof.

Mention may also be made of the mixture of esters of jojoba, of polyglycerin-3, of Acacia decurrens flower wax and of sunflower seed wax, available under the trade name Acticire® from the company Gattefossé. Other examples of suitable apolar waxes are sold by the company Sasol under the trade name Sasol Wax® C80; by the company Jeen under the trade names Jeenate® 2H to 6H; and by the company New Phase under the brand names Performalene® 500 and Performa® V343; another example of suitable apolar wax consists of a mixture of high-molecular-weight linear polyethylene and of ethylene/propylene copolymer available under the brand name Lipwax® PZ80-20 by the company Safic-Alcan.

The waxes according to the invention may also be chosen from micronized waxes.

The waxes according to the invention may also be chosen from silicone waxes, such as C₃₀-C₄₅ alkyl dimethicones, or fluoro waxes.

Composition—Fatty-Phase Gelling Agents

As indicated previously, the composition according to the invention may comprise at least one fatty-phase gelling agent.

Among the fatty-phase gelling agents that can be used in the context of the invention, mention may in particular be made of organopolysiloxane elastomers (also known as silicone elastomers) which can for example be in the form of particles carried in at least one silicone or hydrocarbon-based non-volatile oil, thus forming a gel. This is more particularly a crosslinked silicone elastomer. The elastomer present in the composition according to the invention may be chosen from non-emulsifying or emulsifying elastomers.

The term “emulsifying organopolysiloxane elastomer” is intended to mean an organopolysiloxane elastomer comprising at least one hydrophilic chain, such as polyoxyalkylenated (polyoxyethylenated, polyoxypropylenated) organopolysiloxane elastomers and polyglycerolated silicone elastomers. As polyoxyalkylenated organopolysiloxane elastomers, use may be made of those sold under the names “KSG-21”, “KSG-20”, “KSG-30”, “KSG-31”, “KSG-33”, “KSG-210”, “KSG-310”, “KSG-330”, “KSG-340” by the company Shin Etsu, “DC9010”, “DC9011” by the company Dow Corning. As polyglycerolated organopolysiloxane elastomers, use may be made of those sold under the names “KSG-710”, “KSG-810”, “KSG-820”, “KSG-830”, “KSG-840” by the company Shin Etsu.

As non-emulsifying elastomers, use may for example be made of those sold under the names “DC 9040”, “DC 9041”, “DC 9509”, “DC 9505” by the company Dow Corning; “KSG-6”, “KSG-15”, “KSG-16”, “KSG-18”, “KSG-41”, “KSG-42”, “KSG-43”, “KSG-44” by the company Shin Etsu; Gransil SR SCYC gel, Gransil SR DMF 10 gel, Gransil SR DC556 gel from the company Gransil RPS of Grant Industries; 1229-02-167, 1229-02-168 and “SFE 839” from the company General Electric.

The fatty-phase gelling agents that can be used in the composition according to the invention may be clays that have been modified to make them lipophilic. Mention may be made of hydrophobically modified montmorillonites, such as hydrophobically modified bentonites or hectorites. Mention may for example be made of the product Stearalkonium Bentonite (INCI name) (reaction product of bentonite and of the quaternary ammonium stearalkonium chloride) such as the commercial product sold under the name Tixogel MP 250 by the company Sud Chemie Rheologicals, United Catalysts Inc or the product Disteardimonium Hectorite (INCI name) (reaction product of hectorite and of distearyldimonium chloride) sold under the name Bentone 38 or Bentone Gel by the company Elementis Specialities.

Composition—Pasty Fatty Substances

The composition according to the invention may also comprise at least one compound that is pasty at 25° C. and atmospheric pressure. For the purposes of the present invention the term “pasty” is intended to mean a compound having a reversible solid/liquid change of state, which in the solid state exhibits an anisotropic crystalline arrangement, and comprising, at a temperature of 23° C., a liquid fraction and a solid fraction.

The pasty compound may in particular be chosen from synthetic pasty compounds and fatty substances of plant origin.

The pasty compound(s) may in particular be chosen from:
lanolin and derivatives thereof, such as lanolin alcohol, oxyethylenated lanolins, acetylated lanolin, lanolin esters such as isopropyl lanolate, oxypropylenated lanolins;
petroleum jelly (also known as petrolatum);
polyol ethers chosen from ethers of pentaerythritol and of C₂-C₄ polyalkylene glycol, ethers of fatty alcohol and of sugar, and mixtures thereof;
butters of plant origin, for instance mango butter, such as that sold under the reference Lipex 203 by the company Aarhuskarlshamn, shea butter, in particular that of which the INCI name is Butyrospermum Parkii Butter, such as that sold under the reference Sheasoft((R)) by the company Aarhuskarlshamn, cupuacu butter (Rain Forest RF3410 from the company Beraca Sabara), murumuru butter (Rain Forest RF3710 from the company Beraca Sabara), cacao butter; and also orange wax, for instance that which is sold under the reference Orange Peel Wax by the company Koster Keunen;
phytosterol esters;
triglycerides of fatty acids and derivatives thereof,
pentaerythritol esters;
totally or partially hydrogenated plant oils, for instance hydrogenated soya oil, hydrogenated copra oil, hydrogenated rapeseed oil, mixtures of hydrogenated plant oils, such as the mixture of hydrogenated soya, copra, palm and rapeseed plant oil, for instance the mixture sold under the reference Akogel((R)) by the company Aarhuskarlshamn (INCI name Hydrogenated Vegetable Oil), trans-isomerized partially hydrogenated jojoba oil produced or sold by the company Désert Whale under the commercial reference lso-Jojoba-50(R), partially hydrogenated olive oil, for instance the compound sold under the reference Beurrolive by the company Soliance,
esters of hydrogenated castor oil, such as hydrogenated castor oil dimer dilinoleate, for example Risocast-DA-L sold by Kokyu Alcohol Kogyo, hydrogenated castor oil isostearate, for example Salacos HCIS (V-L) sold by Nisshin Oil;
polymeric or non-polymeric silicone compounds;
polymeric or non-polymeric fluoro compounds;
and mixtures thereof.

Composition—Oils

In addition, the composition preferably comprises 0.01% to 80% by weight relative to the total weight of the composition, more preferentially between 0.5% and 50%, even more preferentially between 1% and 30% of oils.

The oils that can be used may preferably be chosen from hydrocarbon-based or silicone oils, or mixtures thereof.

For the purposes of the present invention, the term “oil” is intended to mean a compound that is liquid at ambient temperature (25° C.) and atmospheric pressure, and that, when it is introduced in a proportion of at least 1% by weight into water at 25° C. is not at all soluble in water, or is soluble in an amount of less than 10% by weight, relative to the weight of oil introduced into the water. The oils that can be used in the present invention may be volatile or non-volatile. The oils may be of plant, mineral or synthetic origin.

Non-Volatile Oils

The oils of plant origin comprise in particular castor oil, sweet almond oil, sunflower oil, rice bran oil, macadamia nut oil, olive oil, wheatgerm oil, groundnut oil, sea buckthorn oil, borage oil, evening primrose oil and palm oil.

The oils of mineral origin comprise in particular paraffin oil and isoparaffin.

The oils of synthetic origin comprise in particular hydrocarbon-based oils such as (poly)ethers or (poly)esters, in particular (poly)esters of C₆ to C₂₀ fatty acids and of C₆ to C₂₀ fatty alcohols which may advantageously be branched, such as diisostearyl malate, dicaprylyl carbonate, isononyl isononanoate; plant oils or derivatives thereof, such as hydrogenated castor oil; branched and/or unsaturated fatty acids; branched and/or unsaturated fatty alcohols; silicone oils such as linear polydimethylsiloxanes (INCI name: Dimethicone) which may optionally be phenylenated, cyclic polydimethylsiloxanes; and mixtures thereof.

When the composition is intended for making up or caring for the lips, it may advantageously comprise at least one glossy oil. The term “glossy oil” is intended to mean an oil that is liquid at 25° C. and atmospheric pressure, characterized by a refractive index of greater than or equal to 1.47. Among the glossy oils that can be used according to the invention, mention may be made of:
hydrocarbon-based oils such as squalane, polybutene, hydrogenated polyisobutene or hydrogenated polydecene,
phenyl silicone oils such as those that can be identified by the INCI names phenyl trimethicone, phenylpropyldimethylsiloxysilicate or trimethyl pentaphenyl trisiloxane,
fluoro silicone oils such as those that can be identified by the INCI name perfluorononyl dimethicone,
aminoceramides such as those that can be identified by the INCI name phytosteryl/octyldodecyl lauroyl glutamate sold in particular by the company Ajinomoto under the trade name Eldew® PS203,
and mixtures thereof.

Volatile Oils

The composition according to the invention may also comprise at least one oil that is volatile at ambient temperature. For the purposes of the invention, the term “volatile oil” is intended to mean an oil capable of evaporating on contact with the skin or the keratin fiber in less than one hour. The volatile oils of the invention are volatile cosmetic oils that are liquid at ambient temperature and that have a non-zero vapor pressure at ambient temperature and atmospheric pressure.

Examples of suitable volatile oils comprise volatile linear alkanes such as those described in document FR2933865 which is incorporated herein by way of reference. Examples of suitable volatile linear alkanes are C₉-C₁₇, in particular C₁₀-C₁₄, alkanes, such as the mixture of undecane and tridecane available under the trade name Cetiol® Ultimate from BASF or the C₁₅-C₁₉ alkanes available under the trade name Emogreen® L15 from SEPPIC, or C₁₂-C₁₄ alkanes available under the trade name Vegelight® 1214 LC from Biosynthis.

It is clearly understood that the composition according to the invention may comprise mixtures of the oils mentioned above.

Composition—Film Forming Agents

The composition according to the invention may also comprise at least one film-forming polymer, in particular capable of introducing staying power and/or non-transfer properties into the composition.

The composition according to the invention may also comprise at least one film-forming agent chosen from hydrocarbon-based and/or silicone-based film-forming agents.

Such a film-forming agent is generally a polymer. This film-forming polymer may be a silicone polymer optionally modified by urethane or fluorine or acrylate, such as the silicone (meth)acrylates sold by Jéen under the name Jéesil PS (which include PS-VH, PS-VHLV, PS-CM, PS-CMLV and PS-DMLV), or the polymers sold by Shin-Etsu under the trade names KP-545, KP-561 and KP-562, or the polymers sold by the company Dow Corning under the trade names Dow Corning® FA 4003 DM, Dow Corning® FA 4002 ID and Dow Corning® FA 4001 CM. Other examples of film-forming polymers are silicone resins and in particular MQ resins such as trimethylsiloxysilicates, and MT resins such as silsesquioxane derivatives and in particular the polymethylsilsesquioxanes sold in particular by the company Shin-Etsu, and also the polypropylsilsesquioxane sold by the company Dow Corning under the trade name Dow Corning® 670 or the phenylpropyl polysilsesquioxane sold by the company Wacker under the trade name Belsil SPR45VP. Another example consists of the fluorosilicone polymers identified by the INCI name trifluoropropyldimethylsiloxy triethylsiloxysilicate, such as that sold by the company General Electric under the trade name XS66-B8226. Use may also be made, as film-forming polymers, of bioadhesive polymers obtained for example by polycondensation of dimethiconol and of MQ silicate resin in a solvent such as heptane, which are in particular sold by the company Dow Corning under the trade names Dow Corning® BIO-PSA 7-4560 Silicone Adhesive, Dow Corning® 7-4405 low tack and Dow Corning® 7-4505 high tack. Other examples of film-forming polymers are cyclic polyolefins such as polycyclopentadiene, in particular sold by the company Kobo under the trade name Koboguard 5400, or else polydicyclopentadiene. Other further examples of film-forming agents consist of copolymers of vinylpyrrolidone (VP) and/or of linear olefins, such as VP/hexadecene and VP/eicosene polymers, including Antaron V216 and Antaron V220 from the company ISP or else ethylene/vinyl acetate copolymers, such as AC 400 from the company Baerlocher. Other film-forming polymers that can be obtained in this invention are polyacrylates such as the poly(ethyl acrylate) sold in particular by the company Creations Couleurs under the trade name Creasil 7 ID.

Composition—Ultraviolet Screening Agents

The cosmetic composition may also comprise 0% to 50% by weight of at least one ultraviolet (UV) screening agent, preferably from 0% to 30%, more preferentially from 0% to 20% by total weight of the composition. In particular, the composition according to the invention may comprise at least one organic or mineral sunscreen or a mixture of the two.

By way of illustration of organic UV screening agents and in a nonlimiting manner, mention may be made of:
anthranilates, in particular menthyl anthranilate;
benzophenones, in particular benzophenone-1, benzophenone-3 or oxybenzone, benzophenone-5, benzophenone-6, benzophenone-8, benzophenone-9, benzophenone-12, and preferentially benzophenone-2 (oxybenzone) or benzophenone-4 (Uvinul MS40® available from BASF);
benzylidenecamphors, in particular 3-benzylidenecamphor, benzylidenecamphorsulfonic acid, camphor benzalkonium methosulfate, polyacrylamidomethylbenzylidenecamphor, terephthalylidenedicamphorsulfonic acid, and preferentially 4-methylbenzylidenecamphor (Eusolex 6300® available from Merck);
benzimidazoles, in particular benzimidazilate (Neo Heliopan AP® available from Haarmann and Reimer), or phenylbenzimidazole sulfonic acid (Parsol HS® available from DSM);
benzotriazoles, in particular drometrizole trisiloxane, or methylene bis-benzotriazolyl tetramethylbutylphenol (Tinosorb M® available from Ciba);
cinnamates, in particular cinoxate, DEA methoxycinnamate, diisopropyl methylcinnamate, glyceryl ethylhexanoate dimethoxycinnamate, isopropyl methoxycinnamate, isoamyl cinnamate, Kaempferia galanga root extract (Tego Galanga from Evonik containing 98% ethyl-p-methoxycinnamate) and preferentially octylmethoxycinnamate (Parsol MCX® available from Hoffmann La Roche);
diphenylacrylates, in particular ethocrylene (Uvinul N35® available from BASF), or octocrylene (Uvinul 539® available from BASF) or ethylhexyl methoxycrylene (Solastay available from Hallstar);
dibenzoylmethanes, in particular butyl methoxydibenzoylmethane (Parsol 1789®);
imidazolines, in particular ethylhexyl dimethoxybenzylidene dioxoimidazoline;
PABAs, in particular ethyl dihydroxypropyl PABA, ethylhexyldimethyl PABA, glyceryl PABA, PABA, PEG-25 PABA, or ethyl PABA (benzocaine);
triazines, in particular anisotriazine (Tinosorb S® available from Ciba) or diethylhexylbutamidotriazone (Uvasorb HEB® available from 3V Sigma), ethylhexyltriazone (Uvinul T150® available from BASF), tris-biphenyl triazine (Tinosorb 2AB available from BASF);
benzoates, in particular N-hexyl 2-(4-diethylamino-2-hydroxybenzoyl)benzoate (Uvinul A+ available from BASF) or as a mixture with octyl methoxycinnamate (Uvinul A+B available from BASF);
benzalmalonates, in particular polysilicone-15 (Parsol SLX available from DSM);
benzoxazoles, in particular 2,4-bis [4-[5-(1,1-dimethylpropyl)benzoxazol-2-yl]phenylimino]-6-[(2-ethylhexyl)imino]-1,3,5-triazine (Uvasorb K2A available from 3V Sigma);
salicylates, in particular dipropylene glycol salicylate, ethylhexyl salicylate, homosalate, butyloctyl salicylate (Hallbrite BHB available from Hallstar) or TEA salicylate.

The inorganic UV screening agents used are, for example, metal oxide particles having an average elementary particle size of less than or equal to 300 nm, preferably less than or equal to 100 nm.

They may in particular be chosen from titanium oxide, zinc oxide, iron oxide, zirconium oxide, cerium oxide or mixtures thereof. The titanium oxides may be in a crystalline form of rutile and/or anatase type, and/or in an amorphous or substantially amorphous form.

The coated titanium oxide pigments may be coated:
with silica (Sunveil from Ikeda);
with silica and iron oxide (Sunveil F from Ikeda);
with silica and polyglyceryl-10 stearate (Cosmeserve WP-40W from Iwase Cosfa);
with silica and alumina (Microtitanium Dioxide MT500 SA and Microtitanium Dioxide MT 100 SA from Tayca, Tioveil from Tioxide);
with alumina (Tipaque TTO-55 (B) and Tipaque TTO-55 (A) from Ishihara, and UVT 14/4 from Kemira);
with alumina-treated rutile TiO₂, and with glycerol-coated silica (UV Titan M212 from Kemira);
with alumina-treated rutile TiO₂ and with dimethicone (UV Titan M195 from Kemira);
with alumina and with aluminum stearate (Microtitanium Dioxide MT 100 T, MT 100 TV, MT 100 TX, MT 100 Z, MT-01 from Tayca, Solaveil CT-10 W and Solaveil CT 100 from Uniqema and Eusolex T-AVO from Merck);
with silica, with alumina and with alginic acid (MT-100 AQ from Tayca);
with alumina and with aluminum laurate (Microtitanium Dioxide MT 100 S from Tayca);
with alumina, with methicone and with polyhydroxystearic acid (INP60T7 from Kobo);
with iron oxide and with iron stearate (Microtitanium Dioxide MT 100 F from Tayca);
with zinc oxide and with zinc stearate (BR 351 from Tayca);
with silica and with alumina and treated with a silicone (Microtitanium Dioxide MT 600 SAS, MICROTITANIUM DIOXIDE MT 500 SAS or Microtitanium Dioxide MT 100 SAS from Tayca);
with silica, with alumina, with aluminum stearate and treated with a silicone (STT-30-DS from Titan Kogyo);
with alumina and treated with a silicone (Tipaque TTO-55 (S) from Ishihara, or UV Titan M 262 from Kemira);
with triethanolamine (STT-65-S from Titan Kogyo);
with stearic acid (Tipaque TTO-55 (C) from Ishihara);
with sodium hexametaphosphate (Microtitanium Dioxide MT 150 W from Tayca);
with octytrimethylsilane-treated TiO₂ (T 805 from the company Degussa Silices);
with polydimethylsiloxane-treated TiO₂ (70250 Cardre UF TiO2S13 by Cardre);
with polydimethylhydrosiloxane-treated anatase/rutile TiO₂ (Microtitanium Dioxide USP Grade Hydrophobic by Color Techniques);
with rutile TiO₂ treated with alumina, stearic acid (UV Titan M160 by Kemira);
with TiO₂ treated with alumina hydroxide, stearic acid and triethoxycaprylylsilane (ALT-T-400 by Maprecos or Titanium Dioxide & Aluminum hydroxide & Stearic acid (ST-705SA/Titan Kogyo));
with manganese-doped TiO₂ (OPT1-PW from Croda).

The uncoated titanium oxide pigments are for example:
Microtitanium Dioxide MT 500 B or Microtitanium Dioxide MT600 B by the company Tayca;
P 25 by the company Degussa;
PW transparent titanium oxide by the company Wacker;
UFTR by the company Miyoshi Kasei;
ITS by the company Tomen;
   and Tioveil AQ by the company Tioxide.

The uncoated zinc oxide pigments are for example:
Z-cote by the company Sunsmart;
Nanox by the company Elementis;
Nanogard WCD 2025 by the company Nanophase Technologies.

The coated zinc oxide pigments are for example:
zinc oxide CS-5 by the company Toshibi (ZnO coated with polymethylhydrosiloxane);
Nanogard Zinc Oxide FN by the company Nanophase Technologies (as a 40% dispersion in Finsolv TN, C₁₂-C₁₅ alkyl benzoates);
Daitopersion ZN-30 and Daitopersion Zn-50 by the company Daito (dispersions in cyclopolymethylsiloxane/oxyethylenated polydimethylsiloxane, containing 30% or 50% of zinc nano oxides coated with silica and polymethylhydrosiloxanes);
NFD Ultrafine ZnO by the company Daikin (ZnO coated with perfluoroalkyl phosphate and copolymer based on perfluoroalkylethyl as a dispersion in cyclopentasiloxane);
SPD-Z1 by the company Shin-Etsu (ZnO coated with silicone-grafted acrylic polymer, dispersed in cyclodimethylsiloxane);
Escalol Z100 by the company ISP (alumina-treated ZnO dispersed in the ethylhexyl methoxycinnamate/PVP-hexadecene copolymer/methicone mixture);
Fuji ZnO-SMS-10 by the company Fuji Pigment (ZnO coated with silica and polymethylsilsesquioxane);
Nanox Gel TN by the company Elementis (ZnO dispersed at 55% in C₁₂-C₁₅ alkyl benzoate with polycondensate of hydroxystearic acid);
OTS-5 MZ-500 by the company Daito (ZnO dispersed in triethoxycaprylylsilane).

The uncoated cerium oxide pigments may for example be those sold under the name Colloidal Cerium Oxide by the company Rhone Poulenc.

Composition—Pigments

The composition according to the invention may comprise from 0% to 50% by weight of at least one pigment.

The term “pigments” should be understood to mean white or colored, mineral and/or organic particles that are insoluble in an aqueous solution and that are intended to color and/or opacify the composition and/or the deposit produced from the composition.

The pigments may be chosen from mineral pigments, organic lakes, nacres, optical-effect pigments, such as reflective particles or interference pigments.

The mineral pigments may be chosen from metal oxide pigments, chromium oxides, iron oxides, titanium dioxide, zinc oxides, cerium oxides, zirconium oxides, manganese violet, Prussian blue, ultramarine blue, ferric blue, ultramarine blue, chromium hydrate and mixtures thereof.

The composition according to the invention may also comprise a particular pigmentary titanium oxide coated with aluminum hydroxide and stearic acid, sold by the company Titan Kogyo under the name ST 705 SA. This pigment has a strong transmittance of rays of red color while at the same time blocking the other rays of the visible spectrum, and increases the sun protection factor.

The cosmetic composition may also comprise 0% to 50%, preferably 0% to 30% by weight, relative to the total weight of the composition, of at least one interference pigment. Interference pigments are pigments capable of generating color by an interference phenomenon. They may consist of a substrate, for example natural mica, silica, synthetic mica, titanium dioxide or glass, around which are deposited one or more layers of a material with a different refractive index, such as titanium dioxide, iron oxide or silica for example.

By way of illustration of interference pigments that may be introduced into the composition, mention may be made of the gold-colored interference pigments in particular sold by the company Engelhard under the name Brilliant gold 212G (Timica), Gold 222C (Cloisonne), Sparkle gold (Timica), Gold 4504 (Chromalite) and Monarch gold 233X (Cloisonne); bronze interference pigments in particular sold by the company Merck under the name Bronze fine (17384) (Colorona) and Bronze (17353) (Colorona) and by the company Engelhard under the name Super bronze (Cloisonne); the orange interference pigments in particular sold by the company Engelhard under the name Orange 363C (Cloisonne) and Orange MCR 101 (Cosmica) and by the company Merck under the name Passion orange (Colorona) and Matte orange (17449) (Microna); the brown-tinted interference pigments in particular sold by the company Engelhard under the name Nu-antique copper 340XB (Cloisonne) and Brown CL4509 (Chromalite); the copper-tinted interference pigments in particular sold by the company Engelhard under the name Copper 340A (Timica); the red-tinted interference pigments in particular sold by the company Merck under the name Sienna fine (17386) (Colorona); the yellow-tinted interference pigments in particular sold by the company Engelhard under the name Yellow (4502) (Chromalite); the gold-tinted red-colored interference pigments in particular sold by the company Engelhard under the name Sunstone G012 (Gemtone); the pink interference pigments in particular sold by the company Engelhard under the name Tan opale G005 (Gemtone); the gold-tinted black interference pigments in particular sold by the company Engelhard under the name Nu-antique bronze 240 AB (Timica), the blue interference pigments in particular sold by the company Merck under the name Matte blue (17433) (Microna), the silvery-tinted white interference pigments in particular sold by the company Merck under the name Xirona Silver and the golden-green pink-orange interference pigments sold in particular by the company Merck under the name Indian summer (Xirona) and mixtures thereof.

The organic lakes are organic pigments formed of a dye attached on a substrate. They may for example be chosen from:
cochenille carmine;
organic pigments of azo dyes, anthraquinone dyes, indigoid dyes, xanthene dyes, pyrene dyes, quinoline dyes, triphenylmethane dyes or fluoran dyes. Among the organic pigments, mention may in particular by made of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6;
insoluble sodium, potassium, calcium, barium, aluminum, zirconium, strontium or titanium salts of acid dyes such as azo, anthraquinone, indigoid, xanthene, pyrene, quinoline, triphenylmethane or fluoran dyes, these dyes possibly comprising at least one carboxylic or sulfonic acid group.

The organic lakes may also be supported by an organic support such as rosin or aluminum benzoate, for example.

Among the organic lakes, mention may in particular be made of those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, FD&C Yellow No. 6 Aluminum lake.

Mention may also be made of liposoluble dyes such as, for example, Sudan red, DC Red 17, DC Green 6, beta-carotene, soybean oil, Sudan brown, DC Yellow 11, DC Violet 2, DC orange 5 or quinolein yellow.

The chemical materials corresponding to each of the abovementioned organic colorants are mentioned in the book “International Cosmetic Ingredient Dictionary and Handbook”, Edition 1997, pages 371 to 386 and 524 to 528, published by “The Cosmetic, Toiletry, and Fragrance Association”, the content of which is incorporated into the present application by way of reference.

Composition—Fillers

The cosmetic composition may also comprise at least one filler, that is to say at least one non-colored insoluble particle. Said filler may be of mineral or organic nature, of any form. Fillers have the effect of modifying the rheology of the composition, the texture, the sensoriality and/or the makeup result produced by the cosmetic composition.

By way of example, mention may be made of talc, mica, silica, kaolin, and polyamide powders, silicone powders, composite powders, and mixtures thereof.

All the compounds in the form of solid particles may have undergone a surface treatment, in particular in order to make them hydrophobic or hydrophilic. The pigments, the mineral screening agents and/or the fillers may be coated after having undergone one or more surface treatments of chemical, electronic, mecanochemical and/or mechanical nature.

The hydrophobic treatment agent may be chosen from silicones, such as methicones, dimethicones, perfluoroalkylsilanes; fatty acids, such as stearic acid; metal soaps, such as aluminum dimyristate, the aluminum salt of hydrogenated tallow glutamate, perfluoroalkyl phosphates, perfluoroalkyl silanes, perfluoroalkyl silazanes, poly(hexafluoropropylene oxide)s, polyorganosiloxanes comprising perfluoroalkyl perfluoropolyether groups, amino acids; N-acylamino acids or salts thereof; lecithin, isopropyl triisostearyl titanate, and mixtures thereof.

The N-acylamino acids may comprise an acyl group having from 8 to 22 carbon atoms, such as for example a 2-ethylhexanoyl, caproyl, lauroyl, myristoyl, palmitoyl, stearoyl or cocoyl group. The salts of these compounds may be the aluminum, magnesium, calcium, zirconium, zinc, sodium or potassium salts. The amino acid may for example by lysine, glutamic acid or alanine.

The term “alkyl” mentioned in the compounds mentioned above denotes in particular an alkyl group having from 1 to 30 carbon atoms, preferably having from 5 to 16 carbon atoms.

Composition—Aqueous Phase

The composition may also comprise one or more aqueous phases. The term “aqueous phase” is intended to mean a phase comprising water and in general any molecule in the dissolved state in the water in the composition.

The aqueous phase of said compositions contains water and in general other water-soluble or water-miscible solvents. The water-soluble or water-miscible solvents comprise monoalcohols with a short chain, for example a C₁-C₄ chain, such as ethanol or isopropanol; humectants such as diols or polyols, for instance ethylene glycol, 1,2-propylene glycol, 1,3-butylene glycol, hexylene glycol, diethylene glycol, dipropylene glycol, and mixtures thereof.

In the case of a simple or multiple emulsion, the aqueous phase may also comprise an emulsifying system. The composition may comprise at least one emulsifier chosen from fatty acid esters of polyols, such as mono-, di-, tri- or sesquioleates or stearates of sorbitol or of glycerol, laurates of glycerol or of polyethylene glycol; silicone surfactants such as alkyl or alkoxy dimethicone copolyols bearing an alkyl or alkoxy chain that is pendent or at the end of the silicone backbone, having from example from 6 to 22 carbon atoms; polymers of polyoxyalkylenated glycol fatty acid ester type. The composition according to the invention comprises an emulsifying surfactant which makes it possible to obtain a water-in-oil emulsion, in particular a surfactant with an HLB (hydrophilic/lipophilic balance) of less than 7. Preferably, the emulsifying surfactant is chosen from fatty acid esters of polyols, such as mono-, di-, tri- or sesquioleates or stearates of sorbitol or of glycerol, laurates of glycerol or of polyethylene glycol; alkyl or alkoxy dimethicone copolyols bearing an alkyl or alkoxy chain that is pendent or at the end of the silicone backbone, having for example from 6 to 22 carbon atoms; polymers of the polyoxyalkylenated glycol fatty acid ester type, alkyl or alkoxy dimethicone copolyols bearing an alkyl or alkoxy chain that is pendent or at the end of the silicone backbone, having for example from 6 to 22 carbon atoms, and mixtures thereof.

The composition may also comprise at least one aqueous-phase gelling agent. The main hydrophilic gelling agents used are crosslinked polymeric gelling agents and natural polymers. As crosslinked polymeric gelling agents, mention may for example be made of carboxyvinyl polymers, such as the products sold under the name Carbopol (INCl name: carbomer) by the company Novéon, polyacrylamides, polymers derived from 2-acrylamido-2-methylpropanesulfonic acid (AMPS) such as the product sold by the company Clariant under the name Hostacerin AMPS, and crosslinked anionic copolymers of acrylamide and of AMPS, such as the product sold under the name Sepigel 305 by the company SEPPIC. As natural polymers, mention may for example be made of xanthan gums and guar gums or else cellulose-based derivatives, starches and alginates. These natural polymers are in general used in combination with the crosslinked polymeric gelling agents since their thickening capacity is most of the time not sufficient for it to be possible to use them alone.

Composition—Usual Cosmetic Ingredients

The cosmetic composition may also comprise additional usual cosmetic ingredients chosen in particular from antioxidants, fragrances, preservatives, neutralizing agents, vitamins, moisturizing agents, self-tanning compounds, antiwrinkle active agents, hydrophilic or lipophilic active agents, free-radical scavengers, deodorizing agents, sequestrants, and mixtures thereof.

Advantageously, the cosmetic composition may be a care composition, a fragrancing composition, or a nonlabial makeup composition.

Of course, the lists of compounds given are not limiting and implicitly comprise the mixtures of the various compounds cited.

It will be possible to understand the invention more clearly by means of the nonlimiting implementation examples described hereinafter, and on examining the appended drawing on which:

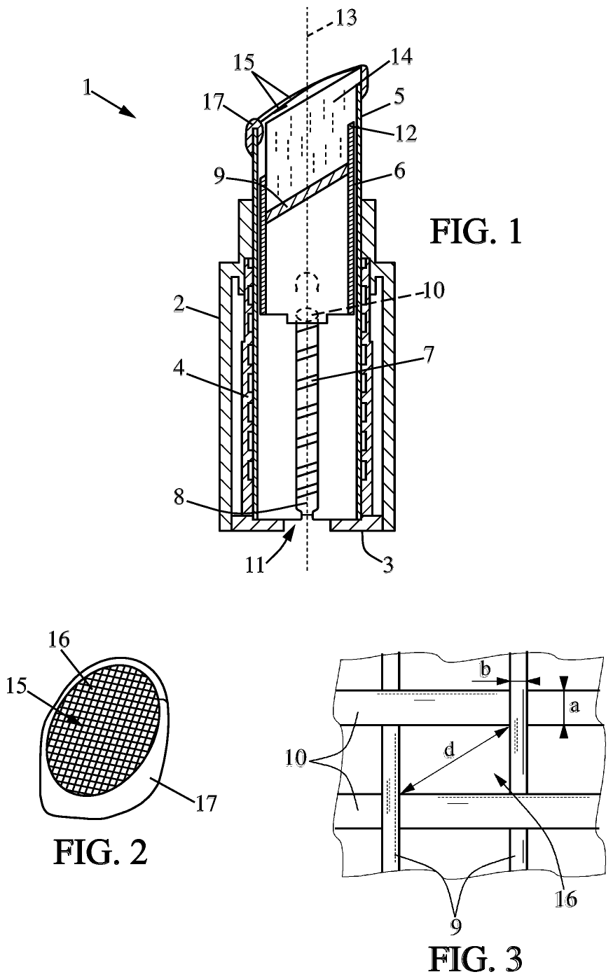

FIG. 1 is a view in longitudinal section of one embodiment of the invention,

FIG. 2 is a perspective view of the grid attached to its support of the device of FIG. 1,

FIG. 3 is an enlarged partial view of FIG. 2, and

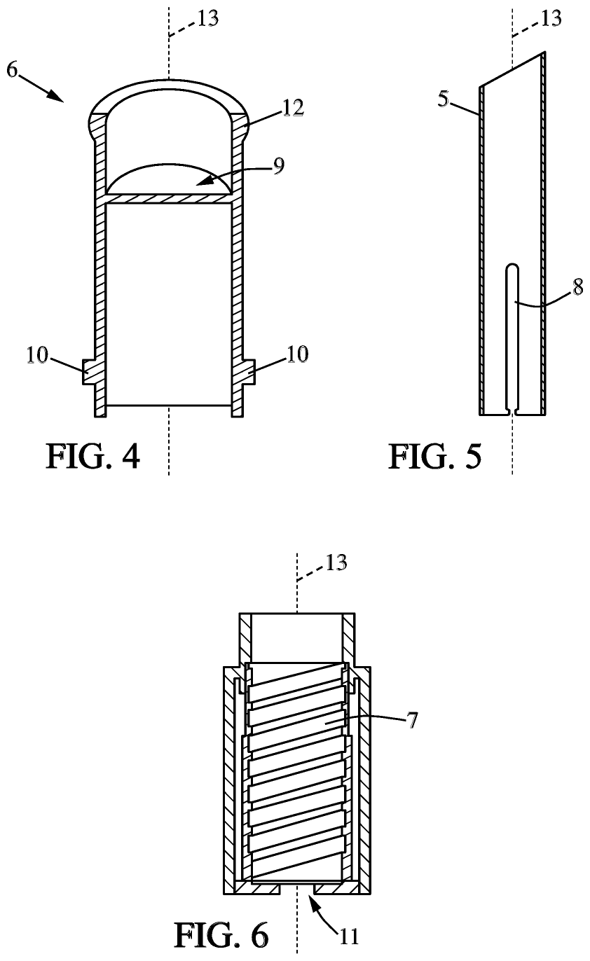

FIGS. 4, 5 and 6 are views in longitudinal section respectively of the piston, of the tube and of the body of the device of FIG. 1.

### DEVICE

The outer shell of the device 1 represented in FIG. 1 is formed by a body 2 and optionally by a cap (not represented). The cap can be reversibly removed. When the cap is in place, it fits onto a part of the body, delimited by a stop, formed by a shoulder, against which it is held by snap-fastening.

The body comprises two axisymmetric portions, preferably along the same axis, and with different cross sections. These two portions are separated by the stop. The body is hollow and open at its distal and proximal ends, which makes it possible to make a housing. The opening 11 of the proximal end of the body is partially blocked by a bottom part 3 of the body which extends, from this end, in a transverse plane, to the body of the applicator.

The housing made in the body 2 comprises means for supporting and moving a cosmetic composition. In particular, the housing advantageously has a piston 6 and a tube 5, the axes 13 of which are the same as the axis of symmetry of the body 2. Optionally, the housing may comprise a tubular element 4 which has a helical ramp, on its outermost part.

The tubular element 4 is detailed in FIG. 6. It is hollow and open at its two ends. It has a substantially smooth external face and an internal face which has a groove forming a helical ramp. This element lies, via its proximal end, on the bottom part 3. The distal end of this element 4 itself extends substantially to the stop. This element is rigidly attached to the body 2.

The tube 5 is detailed in FIG. 5 and forms a hollow cylinder with a circular cross section in a plane perpendicular to the axis 13 and is open at its two ends. The distal end of the tube 5 is for example beveled. The tube 5 can have two indentations, as illustrated, forming two ramps 8 which extend from the proximal end of the tube to a median portion of the tube. The ramps have a general elongated rectilinear shape and are parallel to the axis 13. They are substantially diametrically opposite one another on either side of this axis. In FIGS. 1 and 5, the two ramps are located in symmetrical planes relative to the plane of the sheet, such that only the outline of the ramp in the background is apparent since it is a sectional view. In FIG. 1, the helical ramp 7 is thus apparent through the rectilinear ramp 8. However, in the example illustrated, the helical ramp is indeed carried by the tubular element 4 and the rectilinear ramp by the tube 5.

The piston 6 is detailed in FIG. 4 and forms a hollow cylinder with a substantially circular section in a plane perpendicular to the axis 13. The proximal end of the piston is open and its distal end is beveled and is closed by a portion 9 which is slightly set back relative to the distal end of the piston so as to form a bowl into which is fitted the base of a block of cosmetic composition or baton. This improves the link between the baton and the piston. In FIG. 1, the portion 9 of the piston which closes the distal end is flat and inclined relative to the axis 13 such that it is also beveled.

The piston has, at the level of the external face of its proximal portion, two diametrically opposed guiding blocks 10 which extend, protruding, from the external face of the piston. Of course, it is possible to envisage the blocks being placed at other sites on the external face of the piston. These blocks are each able to travel across both the rectilinear ramp 8 of the tube 5 and the helical ramp 7 of the tubular element 4. Other piston-guiding means may also be suitable.

Optionally, the piston also has, at its distal end, a sealing bead 12 which extends over the entire periphery of the distal end of the piston.

The proximal portion of the tube 5 is then housed within the body and the tubular element 4 and its distal part extends outside, protruding from the body 2 of the device 1. The tube is mounted so as to rotate about the axis 13 relative to the assembly formed by the body 2 and the tubular element 4, by suitable guiding means. The piston 6 is housed so that it moves translationally in the tube 5 by virtue of its blocks 10 capable of travelling across the different ramps 7, 8.

The device 1 also comprises a hard block 14 of cosmetic composition or baton. This baton 14 has a solid cylindrical general shape with a circular cross section in a plane perpendicular to the axis 13. The distal end of the baton 14 has a beveled shape. Of course, it is possible to envisage other shapes of this end, these shapes being widely known to those skilled in the art. The baton 14 rests via its proximal part on the distal end 9 of the piston 6. The piston is thus capable of driving the baton 14 to slide along the axis 13 inside the tube 5.

This baton has a hardness of greater than 20 grams. The hardness is measured according to the protocol subsequently detailed.

The device 1 also comprises a grid 15 with a flat circular general shape. In the embodiment presented, the grid is a woven grid, for example made of metal, comprising warp threads 18 and weft threads 19, these threads forming orifices 16 with a rectangular general shape. The fact that the grid is made of metal is particularly advantageous since this provides, during the application of the composition to the body, a “refreshing” effect that is pleasing for the user. Advantageously, the grid may also be a cloth, preferably woven with a single type of thread. The use of a grid made of textile material makes it possible to obtain flexibility and better comfort on application for the user. A largest dimension d, in this a diagonal, of the orifices 16 is between 50 and 475 μm. The weft and warp threads have a diameter a, b of between 100 and 300 μm which thus also corresponds to the values of minimum distances between the orifices. The grid 15 is rigidly attached to the distal end of the tube 5 via a support 17. In the present example, the orifices are as follows:
largest dimension: 280 μm,
width: 100 μm, 30
length: 261 μm,
warp thread diameter a: 150 μm,
weft thread diameter b: 100 μm,
distance between orifices: 100 μm.

The support 17 has a cylindrical general shape with a circular cross section in a plane perpendicular to the axis 13. The grid 15 is for example linked to the support 17 by overmolding of the support on the grid, the support being made of plastic. The support is linked to the tube 5, this linkage occurring over the whole of the circumference of the distal end of the tube.

As illustrated, the grid(s) are for example oriented substantially along a plane oblique with respect to the axis 13, that is to say not perpendicular to the axis 13. Such an orientation can advantageously make it possible to closely match the beveled shape of the baton. It is also possible to envisage that the grid(s) are oriented substantially perpendicularly to the axis 13.

Operation

When the applicator is not used, the piston 6 is in its lowest position, that is to say that its proximal end is in contact with the part 3 of the device. The baton 14 is located at a distance and facing the grid 15 (not illustrated).

At the time of use, the user holds the device 1 in both hands, their first hand holding the end of the tube 5 protruding out of the body of the device and their other hand holding the body 2 of the device at the level of its portion of largest dimension.

The user applies a rotational movement to the body 2 of the device relative to the tube, about the axis 13. This rotational movement drives the travel of the helical ramp 7 of the tubular element 4 and of the rectilinear ramp 8 of the tube 5 via the two guiding blocks 10 of the piston. This travel drives a translational or rectilinear sliding movement of the piston 6 according to the axis 13, the piston carrying along with it the baton 14. The translational movement continues until the baton 14 comes into contact with the grid 15.

The user continues to apply a rotational movement to the body 2 of the device, thus causing the end of the baton 14 to pass through the grid 15. This passage through the grid leads to a destructuring of an end fraction of the hard baton which is cut into thin slices which subsequently recombine after passing through the grid, out of the grid and over the grid so as to form a homogeneous paste. Once the baton has passed through the grid and has been destructured into a paste, this paste is ready to be applied. The user can thus easily apply this paste to their body, because of its improved spreading properties. Of course, only the end of the block of cosmetic composition is destructured at each application, the largest portion of the baton keeping its integrity. The block is gradually destructured as applications are made, while it rises toward the grid.

In one alternative embodiment that is not represented, the device 1 comprises two grids 15 at the distal end of the tube 5, these two grids being in contact with one another and being attached to the same support 17. The two grids are facing one another and parallel to one another.

At the time of use, the baton passes through the upstream grid then immediately through the downstream grid.

This arrangement has several advantages. Indeed, it makes it possible to have a greater destructuration of the block of cosmetic composition and to thus obtain a more fluid paste.

Furthermore, in particular if the largest dimensions of the orifices 16 of the proximal or upstream grid are greater than the largest dimensions of the orifices 16 of the distal or downstream grid, the effort to be applied in order to cause a portion of the baton to pass through the grids is reduced. The ratio between these two dimensions is greater than 1 and less than or equal to 100, and for example equal to 60.

These two grids 15 may be made of the same material or made of different materials. They may both also have orifices 16 with outlines of different shapes, whether that is within one and the same grid 15 or between the orifices 16 of the two grids. For example, it is possible to envisage a first grid in which the outlines of the orifices have a rectangular shape and a second grid in which the outlines of the orifices have the general shape of an oblong. Of course, a larger number of grids can be envisaged, for example three grids, four grids, five grids or six grids.

Measurement of the Hardness Before Extrusion Through the Grid:

Three cosmetic compositions, numbered 1 to 3, were prepared and their hardnesses were measured before and after extrusion using the cosmetic device according to the invention.

The compositions are presented in table No. 1 below, the amounts are represented as percentage relative to the total weight of the composition. The acronym “qs” (quantity sufficient for) means that it is necessary to add the substance in a proportion sufficient to reach 100% for the total formula.

TABLE 1
cosmetic compositions

| INCI names | Composition 1 | Composition 2 | Composition 3 |
|---|---|---|---|
| SYNTHETIC WAX & ETHYLENE/PROPYLENE | 11-12 | 5-5.5 | — |
| COPOLYMER | | | |
| SYNTHETIC WAX & BHT | 1.5-2 | — | — |
| DIISOSTEARYL MALATE | qs | qs | — |
| DICAPRYLYL CARBONATE | 14-15 | — | — |
| ISONONYL ISONONANOATE | 22-23 | — | — |
| JOJOBA ESTERS & TOCOPHEROL | 4-5 | 5-5.5 | — |
| HYDROGENATED CASTOR OIL | 2-3 | — | — |
| DICAPRYLYL CARBONATE & | 10-11 | — | — |
| STEARALKONIUM HECTORITE & PROPYLENE | | | |
| CARBONATE & TOCOPHEROL | | | |
| ETHYLHEXYL PALMITATE & TRIBEHENIN & | 3.5-4.5 | — | — |
| SORBITAN ISOSTEARATE & PALMITOYL- | | | |
| TRIPEPTIDE-1 | | | |
| BUTYROSPERMUM PARKII | 2 | — | — |
| POLYMETHYLSILSESQUIOXANE | 5 | — | — |
| ALUMINUM STARCH OCTENYLSUCCINATE | 2-3 | — | — |
| DIMETHICONE (100 cSt) | 0-1 | — | — |
| HYDROGENATED COCONUT OIL | — | 16-16.5 | — |
| DIPENTAERYTHRITYL | — | 5-5.5 | — |
| TETRAHYDROXYSTEARATE/ | | | |
| TETRAISOSTEARATE | | | |
| OCTYLDODECANOL | — | 8.5-9.5 | — |
| OCTYLDODECANOL & CERA ALBA | — | 9-10 | — |
| (BEESWAX) | | | |
| POLYBUTENE | — | 20-22 | — |
| STEARYL HEPTANOATE & STEARYL | — | 5.5-6.5 | — |
| CAPRYLATE | | | |
| C20-24 ALKYL DIMETHICONE | — | 0-0.5 | — |
| POLYETHYLENE | — | 2-2.5 | — |
| OCTYLDODECANOL & DISTEARDIMONIUM | — | 6.5-7 | — |
| HECTORITE & PROPYLENE CARBONATE | | | |
| PHYTOSTERYL/OCTYLDODECYL LAUROYL | — | 2-2.5 | — |
| GLUTAMATE | | | |
| CETYL PHOSPHATE | — | — | 1.5 |
| GLYCERIN | — | — | 10 |
| AQUA (WATER) | — | — | 10 |
| ARGININE | — | — | 0.75 |
| DICAPRYLYL CARBONATE | — | — | 17.14 |
| CANDELILLA CERA (EUPHORBIA CERIFERA | — | — | 8 |
| (CANDELILLA) WAX) & BENZYL ALCOHOL | | | |
| JOJOBA ESTERS & POLYGLYCERIN-3 & | — | — | 8 |
| ACACIA DECURRENS FLOWER WAX & | | | |
| HELIANTHUS ANNUUS CERA SEED | | | |
| (HELIANTHUS ANNUUS (SUNFLOWER) SEED | | | |
| WAX) | | | |
| CERA ALBA (BEESWAX) | — | — | 4 |
| SUCROSE TETRASTEARATE TRIACETATE | — | — | 2 |
| BORON NITRIDE | — | — | 1 |
| ETHYLHEXYL METHOXYCINNAMATE & BHT | — | — | 5 |
| SQUALANE | — | — | 6.14 |
| TITANIUM DIOXIDE & STEARIC ACID & | — | — | 7 |
| ALUMINA & SILICA | | | |
| ZINC OXIDE & TRIETHOXYCAPRYLYLSILANE | — | — | 2 |
| DICAPRYLYL CARBONATE | — | — | 6.67 |
| Pigments | 6.5-7.5 | 2-4 | 10.4 |
| Preservatives | 0.5 | 0.5 | 0.4 |
| Fragrances | 0.08 | 0.1 | — |
| TOTAL | 100 | 100 | 100 |

Compositions 1 to 3 are heated so that they are totally in the liquid state. Compositions 1 to 3 of table No. 1 are each poured while hot into three containers, in this case three glass jars with circular walls and a flat, round bottom, of diameter less than 30 mm±0.3 mm. Three samples per composition are then prepared by introducing an amount of cosmetic composition sufficient to obtain a composition thickness of between 8 and 9 mm in the container. The samples are stored at 20° C. for a period of time greater than 24 h before being characterized.

The hardness of each sample is evaluated at 20° C. using a TA-XT Plus Microstable System texurometer from the company Swantech. A stainless steel cylindrical spindle with a diameter of 2 mm penetrates into each sample at a speed of 1 mm·s⁻¹ to a depth of 3 mm, then returns to its initial position after each measurement. Four measurements are carried out on each of the three samples—that is to say twelve measurements in total—by causing the spindle to penetrate in various places.

The hardness value retained for each composition corresponds to the mean of the twelve measurements carried out on the three samples. It is denoted d1 and is expressed in grams (g). The values are reported in table No. 2.

Measurement of the Hardness after Extrusion Through the Grid:

The samples for measuring hardness after extrusion through a grid of the cosmetic device according to the invention are prepared in two steps.

In a first step, compositions 1 to 3 are heated so that they are totally in a liquid state, and poured while hot into the reservoir of a cosmetic article corresponding to an exemplary embodiment of the device according to the invention, fitted with two grids. The first grid upstream of the outlet of the cosmetic composition consists of a cloth woven with a single type of thread having a diameter of 112 μm and openings of on average 160 μm. The second grid downstream of the outlet of the cosmetic composition consists of a cloth woven with a single type of thread, different from the first grid, having a diameter of 36 μm and openings of on average 75 μm. A single sample per composition is prepared, and it is stored for a period of time greater than 24 h at 20° C. before being characterized.

The compositions are then extruded through the two successive grids of the cosmetic device, then transferred into glass jars with circular walls and a flat, round bottom, of diameter less than 30 mm±0.3 mm. The surface of each sample is smoothed using a punch having a smooth, round surface of diameter 30 mm.

The hardness of each sample is evaluated at 20° C. using a TA-XT Plus Microstable System texurometer from the company Swantech. A stainless steel cylindrical spindle with a diameter of 2 mm penetrates into each sample at a speed of 1 mm·s⁻¹ to a depth of 3 mm, then returns to its initial position after each measurement. Four measurements are carried out per sample, by causing the probe to penetrate at various places for each of these four measurements.

The hardness value retained for each of compositions 1 to 3 corresponds to the mean of the four measurements carried out per sample. It is denoted d2 and is expressed in grams (g). The values are reported in table No. 2.

The variation in the hardness of each composition before and after passing through the grids is denoted Δd and is expressed as percentage (%). It is obtained by means of the following formula:

$$Δ d = \frac{( d 2 \times 100 )}{d 1} - 100$$

TABLE 2
values of hardness and of variations in hardness after extrusion
through a first exemplary embodiment of the invention

| | d1 (g) | d2 (g) | Δd (%) |
|---|---|---|---|
| Composition 1 | 246.8 | 15 | −93.9 |
| Composition 2 | 111.7 | 2.4 | −97.8 |
| Composition 3 | 21.4 | 5.8 | −72.9 |

The negative values of Δd indicate that the composition experiences a reduction in its hardness after extrusion.

The three compositions make it possible to cover a wide range of cosmetic compositions of solid or semi-solid type. These results demonstrate that this exemplary embodiment of the cosmetic device makes it possible to obtain a reduction in hardness of at least 73%.

The hardness of composition No. 3, which has the lowest hardness before extrusion (d1), is again measured after extrusion through another set of grids in order to measure the impact of the size of the openings in the cloth constituting the grid. In the same way as previously, composition 3 is poured while hot into the reservoir of a cosmetic device according to the invention, fitted with a first grid upstream of the outlet of the cosmetic composition formed of a cloth woven from a single type of thread having a diameter of 236 μm and openings of 475 μm. The second grid downstream of the outlet of cosmetic composition consists of a cloth woven from a single type of thread, different than the first grid, having a diameter of 90 μm and openings of on average 125 μm. The values of the hardness after extrusion through the device, denoted d2′, and also the variation in hardness after extrusion through this variant of the cosmetic device, Δd′, are given in table No. 3.

TABLE 3
values of hardness and of variations in hardness after extrusion
through a second exemplary embodiment of the invention

| | d1 (g) | d2′ (g) | Δd′ (%) |
|---|---|---|---|
| Composition 3 | 21.4 | 6.6 | −69.1 |

The second exemplary embodiment of a device according to the invention allows a reduction in the hardness of composition No. 3 after extrusion of 70%, which remains satisfactory.

It is understood that the embodiments described are not limiting and that it is possible to introduce improvements into the invention without departing from the context thereof.

Unless otherwise specified, the word “or” is equivalent to “and/or”. Likewise, the term “comprises a” is equivalent to “comprises, inter alia, at least one” unless otherwise specified.

1-16. (canceled)

17. A device for packaging and dispensing a cosmetic composition, comprising:
a housing receiving a cosmetic composition having a hardness of greater than or equal to 20 g; and
at least one grid arranged for the passage of the cosmetic composition through the grid and having through-orifices,
the device being configured such that the cosmetic composition undergoes a reduction of its hardness of greater than 50%, preferably greater than 70%, during the passage of the cosmetic composition through said at least one grid.

18. The device according to claim 17, wherein the through-orifices have a largest dimension of between 40 and 475 μm.

19. The device according to claim 17, wherein the grid or at least one of the grids is woven.

20. The device according to claim 17, comprising at least two grids which follow on from one another in a direction of passage of the cosmetic composition through the grids.

21. The device according to claim 17, wherein the cosmetic composition is in anhydrous form.

22. The device according to claim 17, wherein the cosmetic composition is in the form of a simple or multiple emulsion.

23. The device according to claim 22, wherein the emulsion is an oil-in-water emulsion.

24. The device according to claim 20, wherein the emulsion is a water-in-oil emulsion.

25. The device according to claim 17, wherein the cosmetic composition is packaged in a form chosen from a cast composition, an aqueous gel and an oily gel.

26. The device according to claim 17, wherein the composition comprises 0.5% to 90% by weight of fatty phase-structuring agent chosen from waxes, pasty fatty substances, clays made hydrophobic, and silicone elastomers.

27. The device according to claim 17, wherein the composition comprises 0.01% to 80% by weight of oils that are liquid at 25° C., chosen from hydrocarbon-based and/or silicone oils.

28. The device according to claim 17, wherein the composition comprises at least one film-forming agent chosen from hydrocarbon-based and/or silicone-based film-forming agents.

29. The device according to claim 17, wherein the composition comprises 0% to 50% by weight of at least one ultraviolet screening agent.

30. The device according to claim 17, wherein the composition comprises 0% to 50% by weight of at least one pigment preferably chosen from titanium dioxide, iron oxides, cochenille carmine and organic lakes.

31. The device according to claim 17, wherein the composition comprises 0% to 50% by weight of at least one interference pigment, preferably comprising mica, silica, glass and/or titanium dioxide.

32. The device according to claim 17, wherein the cosmetic composition is a care composition, a fragrancing composition, or a non-labial makeup composition.
